(19) United States
(12) Patent Application Publication
(10) Pub. No.: US 2017/0319661 A1
(43) Pub. Date: Nov. 9, 2017

(54) Methods and Pharmaceutical Compositions Using Orexins (OXA, OXB) for the Treatment of Prostate Cancers

(71) Applicants: INSERM (Institut National de la Sante et de la Recherche Medicale), Paris (FR); Universite de Rouen, Mont-Saint-Aignan (FR); Universite Paris Diderot - Paris 7, Paris (FR)

(72) Inventors: Nicolas Chartrel, Mont Saint Aignan Cedex (FR); Youssef Anouar, Mont Saint Aignan Cedex (FR); Lydie Jeandel, Mont Saint Aignan Cedex (FR); David Alexandre, Mont Saint Aignan Cedex (FR); Jerome Leprince, Mont Saint Aignan Cedex (FR); Alain Couvineau, Paris Cedex 18 (FR); Thierry Voisin, Paris Cedex 18 (FR)

(21) Appl. No.: 15/531,128

(22) PCT Filed: Dec. 3, 2014
(86) PCT No.: PCT/IB2014/002914

§ 371 Date: May 26, 2017

(51) Int. Cl. A61K 38/22; A61K 45/00; G01N 33/574; C12N 15/115; C07K 16/28; A61K 39/00

(52) U.S. Cl. CPC A61K 38/22; C12N 15/115; C07K 16/2869; A61K 45/00; G01N 33/57434; A61K 2039/505; C07K 2319/30; C12N 2310/16; C07K 2317/75; G01N 2500/04; G01N 2800/52

(57) ABSTRACT

The present disclosure relates to methods and pharmaceutical compositions for the treatment of prostate cancers. In particular, the present invention relates to an OX1R agonist for use in the treatment of prostate cancer in a subject in need thereof.

### FIELD OF THE INVENTION

The present invention relates to methods and pharmaceutical compositions for the treatment of prostate cancers, in particular advanced prostate cancer (CaP) and more particularly recurrent androgen-independent prostate cancer (AIPC).

### BACKGROUND OF THE INVENTION

Apart from skin cancer, prostate cancer is the most common form of cancer in men and the second leading cause of cancer deaths in men in the United States (Greenlee, R T et al C A Cancer J. Clin. 50, 7-33 (2000). Initial treatment is usually prostatectomy or radiation to remove or destroy the cancerous cells that are still confined within the prostate capsule. However, many patients are not cured by this therapy and their cancer recurs, or they are diagnosed after the cancer has spread. Tumour growth is initially androgen dependent. Androgen ablation, the mainstay of therapy for progressive prostate cancer, causes regression of androgen-dependent tumours. However, many men eventually fail this therapy and die of recurrent androgen independent prostate cancer (AIPC). AIPC is a lethal form of prostate cancer that progresses and metastasizes. At present, there is no effective therapy for it. There are several pathways by which AIPC can develop. These pathways provide insights into the mechanism of androgen action and schemes by which cancer cells subvert normal growth control and escape attempts to treat the cancer.

Because androgens stimulate tumoral growth, androgen ablation therapy represents at present the main treatment of advanced prostate cancer (CaP) and is initially effective in slowing down the progression of the disease. However, CaP frequently recurs as an androgeno-insensitive tumor with an associated life expectancy of only 15-20 months as no treatment is available until now¹. Prostate tumor cell populations have been reported to be enriched in neuroendocrine cells after a long term anti-androgen therapy²⁻⁴, and it is thought that neuropeptides secreted by the neuroendocrine cells play a crucial role in the progression of the CaP at the advanced AI state. Indeed, bombesin and endothelin-1 have been shown to promote the migration and invasion of CaP cells⁵. NPY stimulates the proliferation of the AI prostate cancer cell line PC3⁶. VIP and PACAP affect the proliferation and neuroendocrine differentiation of LNCaP cells⁷⁻⁹. Adrenomedullin stimulates the proliferation and prevents apoptosis of AI prostate cancer cells^{10,11}, and induces a neuroendocrine phenotype in the AD prostate tumor cells LNCaP¹². Oxytocin has been shown to promote the migration of CaP cells¹³. Finally, it has recently found that the neuropeptide 26RFa stimulates the neuroendocrine differentiation and the migration of AI prostate cancer cells¹⁴.

In contrast, nothing is known about neuropeptides and receptors that may inhibit growth and/or promote apoptosis of prostate cancer cells. Orexins have been reported to be robust stimulants of apoptosis in colon cancer cell lines including HT-29^{15,16} the human neuroblastoma SK-N-MC cells¹⁵ and the rat pancreatic cancer cell line AR42J¹⁷. Furthermore, when colon cancer cells are xenographed in nude mice, treatment with orexins drastically slows down tumor growth, and even reverses the development of established tumors¹⁶. The orexin-driven apoptosis is mediated by the orexin type 1 receptor (OX1R) in colon cancers and neuroblastoma SK-N-MC cells¹⁵ and by the orexin type 2 receptor (OX2R) in pancreatic AR42J cells¹⁷. The same authors also showed that OX1R is aberrantly expressed in primary colorectal tumors as well as in local or distant metastasis, whereas the orexin receptor is absent in normal colonic epithelial cells^{16,18}, opening the way for the use of OX1R agonists for colon cancer therapy.

Therefore, despite marginal advances in prostate cancer treatment, there remains a need for improved therapies and more creative approaches to devising and delivering effective prostate cancer therapies.

The orexins (hypocretins) comprise two neuropeptides produced in the hypothalamus: the orexin A (OX-A) (a 33 amino acid peptide) and the orexin B (OX-B) (a 28 amino acid peptide) (Sakurai T. et al., Cell, 1998, 92, 573-585). Orexins are found to stimulate food consumption in rats suggesting a physiological role for these peptides as mediators in the central feedback mechanism that regulates feeding behaviour. Orexins regulate states of sleep and wakefulness opening potentially novel therapeutic approaches for narcoleptic or insomniac patients. Orexins have also been indicated as playing a role in arousal, reward, learning and memory. Two orexin receptors have been cloned and characterized in mammals. They belong to the super family of G-protein coupled receptors (7-transmembrane spanning receptor) (Sakurai T. et al., Cell, 1998, 92, 573-585): the orexin-1 receptor (OX1R or HCTR1) is selective for OX-A and the orexin-2 receptor (OX2R or HCTR2) is capable to bind OX-A as well as OX-B.

### SUMMARY OF THE INVENTION

The present invention relates to methods and pharmaceutical compositions for the treatment of prostate cancers. In particular, the present invention relates to an OX1R agonist for use in the treatment of prostate cancer in a subject in need thereof.

### DETAILED DESCRIPTION OF THE INVENTION

The present invention relates to a OX1R agonist for use in the treatment of prostate cancer in a subject in need thereof.

As used herein, the tem “OX1R” has its general meaning in the art and refers to the 7-transmembrane spanning receptor OX1R for orexins. According to the invention, OX1R promotes apoptosis in the human prostatic cancer cell line through a mechanism which is not related to Gq-mediated phopholipase C activation and cellular calcium transients. Orexins induce indeed tyrosine phosphorylation of 2 tyrosine-based motifs in OX1R, ITIM and ITSM, resulting in the recruitment of the phosphotyrosine phosphatase SHP-2, the activation of which is responsible for mitochondrial apoptosis (Voisin T, El Firar A, Rouyer-Fessard C, Gratio V, Laburthe M. A hallmark of immunoreceptor, the tyrosine-based inhibitory motif ITIM, is present in the G protein-coupled receptor OX1R for orexins and drives apoptosis: a novel mechanism. FASEB J. 2008 June; 22(6):1993-2002; El Firar A, Voisin T, Rouyer-Fessard C, Ostuni M A, Couvineau A, Laburthe M. Discovery of a functional immunoreceptor tyrosine-based switch motif in a 7-transmembrane-spanning receptor: role in the orexin receptor OX1R-driven apoptosis. FASEB J. 2009 December; 23(12):4069-80. doi: 10.1096/fj.09-131367. Epub 2009 Aug. 6.). An exemplary amino acid sequence of OX1R is shown as SEQ ID NO:1.

| orexin receptor-1 OX1R | |
|---|---|
| (SEQ ID NO: 1) | |
| 1 | mepsatpgaq mgvppgsrep spvppdyede flrylwrdyl ypkqyewvli aayvavfvva |
| 61 | lvgntlycla vwrnhhmrtv tnyfivnlsl advlvtaicl pasllvdite swlfghalck |
| 121 | vipylqavsv svavltlsfi aldrwyaich pllfkstarr argsilgiwa vslaimvpqa |
| 181 | avmecssvlp elanrtrlfs vcderwaddl ypkiyhscff ivtylaplgl mamayfqifr |
| 241 | klwgrqipgt tsalvrnwkr psdqlgdleq glsgepqprg raflaevkqm rarrktakml |
| 301 | mvvllvfalc ylpisvinvl krvfgmfrqa sdreavyacf tfshwlvyan saanpiiynf |
| 361 | lsgkfreqfk aafscclpgl gpcgslkaps prssashksl slqsrcsisk isehvvltsv |
| 421 | ttvlp |

Accordingly, as used herein, the term “OX1R agonist” refers to any compound natural or not that is able to bind to OX1R and promote OX1R activity which consists of activating signal transduction pathways involving recruitment of SHP-2 and inducting apoptosis of the cell, independently on transient calcium release.

In some embodiments, the OX1R agonist is a small organic molecule. The term “small organic molecule” refers to a molecule of a size comparable to those organic molecules generally used in pharmaceuticals. The term excludes biological (macro)molecules (e.g., proteins, nucleic acids, etc.). Preferred small organic molecules range in size up to about 5000 Da, in particular up to 2000 Da, and preferably up to about 1000 Da.

In some embodiment, the OX1R agonist is a OX1R antibody or a portion thereof.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

In one embodiment, the antibody or portion thereof is a monoclonal antibody. In one embodiment, the antibody or portion thereof is a polyclonal antibody. In one embodiment the antibody or portion thereof is a humanized antibody. In one embodiment, the antibody or portion thereof is a chimeric antibody. In one embodiment, the portion of the antibody comprises a light chain of the antibody. In one embodiment, the portion of the antibody comprises a heavy chain of the antibody. In one embodiment, the portion of the antibody comprises a Fab portion of the antibody. In one embodiment, the portion of the antibody comprises a F(ab′)2 portion of the antibody. In one embodiment, the portion of the antibody comprises a Fc portion of the antibody. In one embodiment, the portion of the antibody comprises a Fv portion of the antibody. In one embodiment, the portion of the antibody comprises a variable domain of the antibody. In one embodiment, the portion of the antibody comprises one or more CDR domains of the antibody.

Antibodies are prepared according to conventional methodologies. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of OX1R. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. Briefly, the recombinant OX1R may be provided by expression with recombinant cell lines. In particular, OX1R may be provided in the form of human cells expressing OX1R at their surface. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3 A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., I. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and portions thereof may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

In one embodiment of the agents described herein, the OX1R agonist is a polypeptide. In a particular embodiment the polypeptide is a functional equivalent of Orexin-A or Orexin-B.

As used herein the term “orexin-A” has its general meaning in the art and refers to the amino acid sequence as shown by SEQ ID NO:2.

Orexin-A (SEQ ID NO: 2):
ₚeplpdccrqk tcscrlyell hgagnhaagi ltl

ₚe means pyroglutamate

As used herein the term “orexin-B” has its general meaning in the art and refers to the amino acid sequence as shown by SEQ ID NO:3.

Orexin-B (SEQ ID NO: 3):
l fsgppglqgr lqrllqasgn haagiltm

As used herein, a “functional equivalent of orexin” is a polypeptide which is capable of binding to OX1R, thereby promoting an OX1R activity according to the invention. The term “functional equivalent” includes fragments, mutants, and muteins of Orexin-A and Orexin-B. The term “functionally equivalent” thus includes any equivalent of orexins (i.e. Orexin-A or Orexin-B) obtained by altering the amino acid sequence, for example by one or more amino acid deletions, substitutions or additions such that the protein analogue retains the ability to bind to OX1R and promote an OX1R activity according to the invention (e.g. aoptosis of the cancer cell). Amino acid substitutions may be made, for example, by point mutation of the DNA encoding the amino acid sequence.

In some embodiments, the functional equivalent is at least 80% homologous to the corresponding protein. In a preferred embodiment, the functional equivalent is at least 90% homologous as assessed by any conventional analysis algorithm such as for example, the Pileup sequence analysis software (Program Manual for the Wisconsin Package, 1996). The term “a functionally equivalent fragment” as used herein also may mean any fragment or assembly of fragments of Orexin that binds to OX1R and promote the OX1R activity according to the invention. Accordingly the present invention provides a polypeptide which comprises consecutive amino acids having a sequence which corresponds to the sequence of at least a portion of Orexin-A or Orexin-B, which portion binds to OX1R and promotes the OX1R activity according to the invention.

Functionally equivalent fragments may belong to the same protein family as the human Orexins identified herein. By “protein family” is meant a group of proteins that share a common function and exhibit common sequence homology. Homologous proteins may be derived from non-human species. In particular, the homology between functionally equivalent protein sequences is at least 25% across the whole of amino acid sequence of the complete protein. More In particular, the homology is at least 50%, even more In particular 75% across the whole of amino acid sequence of the protein or protein fragment. More In particular, homology is greater than 80% across the whole of the sequence. More In particular, homology is greater than 90% across the whole of the sequence. More In particular, homology is greater than 95% across the whole of the sequence.

The polypeptides of the invention may be produced by any suitable means, as will be apparent to those of skill in the art. In order to produce sufficient amounts of polypeptides or functional equivalents thereof for use in accordance with the present invention, expression may conveniently be achieved by culturing under appropriate conditions recombinant host cells containing the polypeptide of the invention. In particular, the polypeptide is produced by recombinant means, by expression from an encoding nucleic acid molecule. Systems for cloning and expression of a polypeptide in a variety of different host cells are well known. When expressed in recombinant form, the polypeptide is in particular generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E. coli.

In one embodiment, the polypeptide of the invention is an immunoadhesin.

As used herein, the term “immunoadhesin” designates antibody-like molecules which combine the binding specificity of a heterologous protein (an “adhesin” which is able to bind to OX1R) with the effector functions of immunoglobulin constant domains. Structurally, the immunoadhesins comprise a fusion of an amino acid sequence with the desired binding specificity to OX1R (i.e., is “heterologous”), and an immunoglobulin constant domain sequence. The adhesin part of an immunoadhesin molecule typically is a contiguous amino acid sequence comprising at least the binding site for OX1R. In one embodiment, the adhesin comprises the polypeptides characterized by SEQ ID NO:2 or SEQ ID NO:3. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The immunoglobulin sequence typically, but not necessarily, is an immunoglobulin constant domain (Fc region). Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use.

In one embodiment, the Fc region is a native sequence Fc region. In one embodiment, the Fc region is a variant Fc region. In still another embodiment, the Fc region is a functional Fc region. As used herein, the term “Fc region” is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The adhesion portion and the immunoglobulin sequence portion of the immunoadhesin may be linked by a minimal linker. The immunoglobulin sequence typically, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but typically IgG1 or IgG3.

The polypeptides of the invention, fragments thereof and fusion proteins (e.g. immunoadhesin) according to the invention may exhibit post-translational modifications, including, but not limited to glycosylations, (e.g., N-linked or O-linked glycosylations), myristylations, palmitylations, acetylations and phosphorylations (e.g., serine/threonine or tyrosine).

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In one embodiment, the OX1R agonist is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods.

The term “prostate cancer>> as used herein relates to cancer which is derived from prostate cells. In particular, prostate cancer included prostate adenocarcinoma prostate neuroendocrine tumors in particular advanced prostate cancer (CaP) and more particularly recurrent androgen-independent prostate cancer (AIPC).

In some embodiments, the OX1R agonist of the invention is administered to the subject in a therapeutically effective amount.

By a “therapeutically effective amount” is meant a sufficient amount of OX1R to treat prostate cancer at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The OX1R agonist of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

In particular, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The OX1R agonist of the invention may be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier may also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifuCASK agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various other ingredients listed above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The OX1R agonist of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

In some embodiments, the OX1R agonist of the invention is used in combination with a chemotherapeutic agent. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifo sfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

A further aspect of the invention is a method for treating prostate cancer in a subject in thereof comprising the steps consisting of i) determining the expression level of OX1R in a tumour tissue sample obtained from the subject, ii) comparing the expression level determined at step i) with a reference value and iii) administering the subject with a therapeutically effective amount of a OX1R agonist when the level determined at step i) is higher than the reference value.

The expression level of OX1R may be determined by any well known method in the art. For example methods for determining the quantity of mRNA are well known in the art. Typically the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous. Alternatively an immunohistochemistry (IHC) method may be used. IHC specifically provides a method of detecting targets in a sample or tissue specimen in situ. The overall cellular integrity of the sample is maintained in IHC, thus allowing detection of both the presence and location of the targets of interest (i.e. OX1R). Typically a sample is fixed with formalin, embedded in paraffin and cut into sections for staining and subsequent inspection by light microscopy. Current methods of IHC use either direct labeling or secondary antibody-based or hapten-based labeling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan). In particular embodiment, a tumor tissue section may be mounted on a slide or other support after incubation with antibodies directed against OX1R. Then, microscopic inspections in the sample mounted on a suitable solid support may be performed. For the production of photomicrographs, sections comprising samples may be mounted on a glass slide or other planar support, to highlight by selective staining the presence of the proteins of interest.

A “reference value” may be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Typically, the threshold value is derived from the OX1R expression level (or ratio, or score) determined in a tumor tissue sample derived from one or more subjects having sufficient amount of OX1R level to get an efficient treatment with the OX1R agonist. Furthermore, retrospective measurement of the OX1R expression levels (or ratio, or scores) in properly banked historical subject samples may be used in establishing these threshold values.

A further aspect of the invention is a method for screening a drug for the treatment of prostate cancer comprising the steps of i) providing a plurality of test substances ii) determining whether the test substances are OX1R agonists and iii) positively selecting the test substances that are OX1R agonists.

Typically, the screening method of the invention involves providing appropriate cells which express the orexin-1 receptor on their surface. Such cells include cells from mammals, yeast, Drosophila or E. coli. In particular, a polynucleotide encoding the orexin-1 receptor is used to transfect cells to express the receptor. The expressed receptor is then contacted with a test substance and an orexin-1 receptor ligand (e.g. orexins), as appropriate, to observe activation of a functional response such as recruitment of SHP-2 and induction of cell apoptosis of the cell. Functional assays may be performed as described in El Firar A, Voisin T, Rouyer-Fessard C, Ostuni M A, Couvineau A, Laburthe M. Discovery of a functional immunoreceptor tyrosine-based switch motif in a 7-transmembrane-spanning receptor: role in the orexin receptor OX1R-driven apoptosis. FASEB J. 2009 December; 23(12):4069-80. doi: 10.1096/fj.09-131367. Epub 2009 Aug. 6. In particular comparison steps may involve to compare the activity induced by the test substance and the activity induce by a well known OX1R agonist such as orexin. In particular substances capable of having an activity similar or even better than a well known OX1R agonist are positively selected.

Typically, the screening method of the invention may also involve screening for test substances capable of binding of to orexin-1 receptor present at cell surface. Typically the test substance is labelled (e.g. with a radioactive label) and the binding is compared to a well known OX1R agonist such as orexin. The preparation is incubated with labelled OX1R and complexes of test substances bound to NGAL are isolated and characterized according to routine methods known in the art. Alternatively, the OX1R may be bound to a solid support so that binding molecules solubilized from cells are bound to the column and then eluted and characterized according to routine methods. In another embodiment, a cellular compartment may be prepared from a cell that expresses a molecule that binds NGAL such as a molecule of a signalling or regulatory pathway modulated by NGAL. The preparation is incubated with labelled NGAL in the absence or the presence of a candidate compound. The ability of the candidate compound to bind the binding molecule is reflected in decreased binding of the labelled ligand.

Typically, the candidate compound is selected from the group consisting of small organic molecules, peptides, polypeptides or oligonucleotides.

The test substances that have been positively selected may be subjected to further selection steps in view of further assaying their properties for the treatment of prostate cancer. For example, the candidate compounds that have been positively selected may be subjected to further selection steps in view of further assaying their properties in animal models of prostate cancer.

The above assays may be performed using high throughput screening techniques for identifying test substances for developing drugs that may be useful to the treatment of prostate cancer. High throughput screening techniques may be carried out using multi-well plates (e.g., 96-, 389-, or 1536-well plates), in order to carry out multiple assays using an automated robotic system. Thus, large libraries of test substances may be assayed in a highly efficient manner. More particularly, stably-transfected cells growing in wells of micro-titer plates (96 well or 384 well) can be adapted to high through-put screening of libraries of compounds. Compounds in the library will be applied one at a time in an automated fashion to the wells of the microtitre dishes containing the transgenic cells described above. Once the test substances which activate the apoptotic signals are identified, they can be positively selected for further characterization. These assays offer several advantages. The exposure of the test substance to a whole cell allows for the evaluation of its activity in the natural context in which the test substance may act. Because this assay can readily be performed in a microtitre plate format, the assays described can be performed by an automated robotic system, allowing for testing of large numbers of test samples within a reasonably short time frame. The assays of the invention can be used as a screen to assess the activity of a previously untested compound or extract, in which case a single concentration is tested and compared to controls. These assays can also be used to assess the relative potency of a compound by testing a range of concentrations, in a range of 100 μM to 1 μM, for example, and computing the concentration at which the apoptosis is maximal.

The invention will be further illustrated by the following figures and examples. However, these examples and figures should not be interpreted in any way as limiting the scope of the present invention.

### FIGURES

FIG. 1. Representative microscopic fields of prostate tumors at various grades showing the immunocytochemical distribution of the orexin type 1 (OX1R) receptor, and the neuroendocrine marker EM66. (A) Photomicrograph of an adenocarcinomatous formation in a Gleason's score (4+3) CaP showing OX1R-like immunoreactivity (LI). (B) Cancerous mass in a Gleason's score (3+3) CaP where malignant cells infiltrate the acinar lumen. Some of the invasive cells show OX1R-LI while others are totally unstained. (C) Section of a Gleason's score (4+5) CaP showing a well-defined cancerous formation strongly labelled with the OX1R antibodies. (D) Higher magnification of C reveals that most of the cancerous cells display OX1R-LI. (E and F) Consecutive sections of a Gleason's score (4+5) CaP showing that OX1R (E) and EM66 (F) antibodies label the same adenocarcinomatous structures. Scale bars: 100 μm (A, C, E, F); 50 μm (B and D).

FIG. 2. Representative microscopic fields of prostate tumors at various grades showing the immunocytochemical distribution of orexin A (OxA), α-actin and PGP 9.5. (A) Section of a Gleason's score (4+3) CaP treated with OxA antibodies showing a cancerous formation totally unstained. (B) Photomicrogaph of a Gleason's score (3+3) CaP showing the presence of OxA-immunoreactive “fiber like” structures in the stroma of the gland. (C and D) Consecutive sections of a Gleason's score (3+3) CaP showing that OxA antibodies (C) and α-actin antibodies (D; a specific marker of smooth muscle fibers) do not label the same structures. (E and F) Consecutive sections of a Gleason's score (3+3) CaP showing that OxA antibodies (E) and PGP 9.5 antibodies (F; a specific marker of nerve fibers) do not label the same structures. Scale bars: 100 μm.

FIG. 3. Quantification of OX1R-immunoreactive cells in benign prostate hyperplasia and prostate tumor sections at various stages. The number of OX1R-immunoreactive cells is significantly higher in the CaP sections as compared to the BPH sections whatever the grade of the cancer. In addition, the percentage of cancer cells labelled with the OX1R antibodies increases with the grade of the CaP. Values are mean±SEM of 5 determinations performed on 20 distinct sections of BPH and CaP at various stages. Data were analyzed by using the Mann-Whitney U test. *, p<0.05; ***, p<0.001; ns, not significant.

FIG. 4. Expression of OX1R mRNAs in prostate cancer cell lines. OX1R mRNA levels were determined by quantitative PCR and adjusted to the signal intensity of HPRT1 in two distinct prostate cancer cell lines, i.e. LNCaP cells which are androgeno-dependent, and DU145 cells which are androgeno-independent. (A) The OX1R gene is expressed in the androgeno-independent DU145 cell line, but not in the androgeno-dependent LNCaP cells. (B) Induction of a neuroendocrine differentiation, by addition of db-cAMP (1 mM)/IBMX (0.1 mM) in the culture medium, induces a significant increase of OX1R mRNA expression in the DU145 cells, but does not promote the expression of the OX1R gene in the LNCaP cells. (C) Protein expression assessed by Western blot on whole lysates of DU145 cells confirms the up-regulation of OX1R in DU145 cells exhibiting a neuroendocrine phenotype. Equal protein loading was determined by reprobing with antibodies to α-tubulin. Values are mean±SEM of 5 determinations in four independent experiments for each cell line. Data were analyzed by using the Mann-Whitney U test. ***, p<0.001.

FIG. 5. Effects of orexin A and B on the apoptosis of native and trans-differentiated DU145 cells. Impact of orexin A and B (OxA and OxB; 10-6 M) on the apoptosis level of the androgeno-independent DU145 cells, submitted or not to a neuroendocrine differentiation (by addition of db-cAMP (1 mM)/IBMX (0.1 mM)), was assessed by using an Apo-ONE Homogeneous Caspase3/7 assay. (A and B) Addition of OxA or OxB in the culture medium has no effect on the apoptosis rate of native DU145 cells. (C and D) When the DU145 cells are submitted to a neuroendocrine differentiation, orexins induce a significant increase of the cell apoptosis. (E and F) In the same set of experiments, the effects of OxA and OxB on the HT-29 cell line, used as a positive control, were investigated. As previously described, orexins strongly promote the apoptosis of the HT-29 cells. Values are mean±SEM of 4 determinations in 10 independent experiments. *, p<0.05; **, p<0.01; ***, p<0.001; ns, non significant.

FIG. 6 Effect of inoculation of orexin-A on the growth of tumors developed by xenografting human DU-145 cells in nude mice—DU-145 cells were inoculated in the flank of nude mice at day 0. Left, Mice were injected (2 injections/week) intraperitoneally with 100 μl of orexin-A solutions (0.22 μmoles of orexin-A/Kg) starting at day 1 (white circles) or day 26 (white triangles) or with 100 μl of PBS (black circles) for controls. Right, after 45 days of treatment, mice were sacrificed and tumor volume and weight were then challenged. The development of tumors was followed by calliper measurement. Data are means±SE of 8 tumors in each group. *** p<0.01 versus control.

### EXAMPLE

### Example 1. Material & Methods

Immunohistochemical Procedure

Deparaffinized sections (15-μm thick) from 5 BPH and 15 prostate tumors at various stages (Gleason scores: 3+3, 4+3 and 4+5) were obtained from the Department of Pathology of the University Hospital of Rouen. CaP sections were incubated for 1 h at room temperature with rabbit polyclonal antibodies against orexin A (#AB3704, Millipore, Billerica, Mass.) diluted 1:1000, or to OX1R (#PAB8017, Abnova, Taipan, Taiwan) diluted 1:250, or to OX2R (#OX2R22-A, Alpha Diagnostic International Inc, San Antonio, Tex.) diluted 1:250, or to EM66 19 diluted 1:600, or to α-actin (#AB5694, Abcam, Paris, France) diluted 1:500, or to protein gene product PGP9.5 (# AB1761, Chemicon International, Temecula, Calif.) diluted 1:250. The sections were incubated with a streptavidin-biotin-peroxydase complex (Dako Corporation, Carpinteria, Calif.), and the enzymatic activity was revealed with diaminobenzidine. The slices were then counterstained with hematoxylin. Observations were made under a Nikon E 600 light microscope.

The specificity of the immunoreactions was controlled by (1) substitution of the primary antibodies with Tris buffer saline (TBS; pH 7.4) and (2) preincubation of the orexin A antiserum (diluted 1:1000) with synthetic human orexin A (10-6 M; Tocris Bioscience, Bristol, UK), or preincubation of the EM66 antiserum (diluted 1:600) with recombinant human EM66 (10-6 M).

OX1R-immunoreactive cells present in the adenocarcinomatous masses (for the CaP sections) or the acini (for the BPH sections) were quantified. For this, 5 independent fields of 20 distinct sections of BPH or prostate tumors were photographed at a 20× magnification. The number of immunostained cells present in each image was evaluated by using the cell counter plugin of the image analysis software Image J, and expressed as a percentage of the total number of cells present on the photomicrograph.

Cancer Cell Lines

Two human-derived CaP cell lines were used, i.e. the androgen-responsive cell line LNCaP, and the androgen-unresponsive cell line DU145 as well as the colorectal adenocarcinoma HT-29 cell line. The three cell lines were purchased from American Type Culture Collection (ATCC, Rockville, Md.). The LNCaP cells were maintained in RPMI 1640 medium supplemented with 10% fetal bovine serum (FBS), 1% penicillin/streptomycin and 1% glutamine (complete medium). The DU145 cells were maintained in Dulbecco modified Eagle's minimal essential medium (DMEM) supplemented with 10% FBS and 1% penicillin/streptomycin (complete medium). The HT-29 cells were maintained in DMEM supplemented with 10% FBS, 1% penicillin/streptomycin and 1% glutamine. The cells were grown at 37° C. in a humidified 95% air/5% CO2 atmosphere and the culture medium was replaced every 3 days. When the cells reached 70-80% confluence, they were washed with phosphate buffer saline (PBS; pH 7.4), harvested by a brief incubation with 0.25% trypsin-EDTA solution, and seeded as suggested by ATCC.

RNA Extraction, Reverse Transcription and Quantitative PCR

Total RNA from cell lines was extracted with the Tri-reagent (Sigma-Aldrich, Lyon, France), purified by using a Nucleospin kit (Macherey-Nagel, Hoerdt, France), and quantified with a Nanodrop spectrometer (Nanodrop Technologies, Wilmington, Del.). Contaminating genomic DNA was removed by treatment with deoxyribonuclease I, and cDNAs were synthesized from 1 to 5 μg RNA using the ImProm II Reverse Transcriptase (Promega Corp., Madison, Wis.). Quantitative PCR was performed by using the 7900 HT Fast Real-time PCR System and Gene Expression Master Mix 2× assay (Applied Biosystems, Courtaboeuf, France). OX1R and hypoxanthine ribosyltransferase (HPRT1) primers were designed by using Primer Express software version 3.0: HPRT1 forward primer 5′-GACTTTGCTTTCCTTGGTCAGGCA-3′(SEQ ID NO:4); HPRT1 reverse primer 5′-ACAATCCGCCCAAAGGGAACTGA-3′(SEQ ID NO:5); OX1R forward primer 5′-GTGGGCAACACGCTGGTCTG-3′(SEQ ID NO:6); OX1R reverse primer 5′-GGCCCCAGAGCTTGCGGAAT-3′(SEQ ID NO:7). The purity of the PCR products was assessed by dissociation curves. The amount of target cDNA was calculated by the comparative threshold (Ct) method and expressed by means of the 2-ΔΔCt method according to Applied Biosystems instructions using HPRT1 as an internal control. Expression of HPRT1 mRNA was not affected by treatments or the nature of the tissues, and the ratio of ΔCt value did not vary with the amount of cDNA.

Neuroendocrine Differentiation of DU145 Cells

DU145 cells were seeded at a density of 5×104 cells/well in 12-well plates and starved 24 h in minimum medium (complete medium in which 10% FBS was replaced by 1% FBS). Neuroendocrine differentiation of the cells was carried out by adding to the culture medium 1 mM dituryl-cyclic adenosine monophosphate (db-cAMP)/0.1 mM 3-isobutyl-1-methylxanthine (IBMX). Medium was changed every day until day 5.

Ability of db-cAMP/IBMX to induce a neuroendocrine differentiation of the DU145 cells was assessed by quantifying the increase of expression of three markers of neuroendocrine differentiation, i.e. chromograninA, secretogranin II and neurone specific enolase, and the occurrence of neurite-like extensions as previously described14.

Viability and Apoptosis of DU145 Cells

Cell viability was determined by using the CellTiter-Blue® assay (Promega, Charbonnières, France). Cell apoptosis was quantified by evaluating the enzymatic activities of Caspase 3/7 by using the Apo-ONE® Homogeneous Caspase-3/7 Assay (promega). Briefly, the enzymes were measured in intact and differentiated DU145 culture cells after a 3-days incubation with OxA or OxB (10-6M) in 96-well plates. The cells were incubated with fluorogenic peptide specific to caspase 3 and 7 enzymes, and detection of fluorescent product over time was monitored in a spectrofluorometer Flex Station 3 (Molecular Devices, St. Grégoire, France) at 485 nm excitation and 527 nm emission. Data were normalized to equivalent cell numbers per treatment group.

Statistical Analysis

All of the experiments were performed in triplicate and repeated at least three times. Results are expressed as mean±SEM. All statistical analyses were performed with GraphPad Prism 4 data analysis software. The Mann-Whitney U test was used for comparison of the mean values between two groups. Differences were considered statistically significant at *: p<0.05, **: p<0.01, ***: p<0.001.

### Example 2. Results

2.1. Immunohistochemical Distribution of Orexin Type 1 Receptor and Orexin A in Prostate Tumors

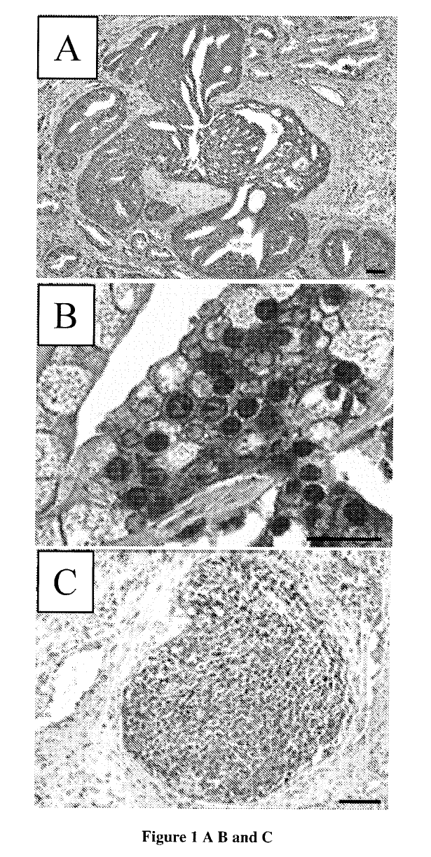

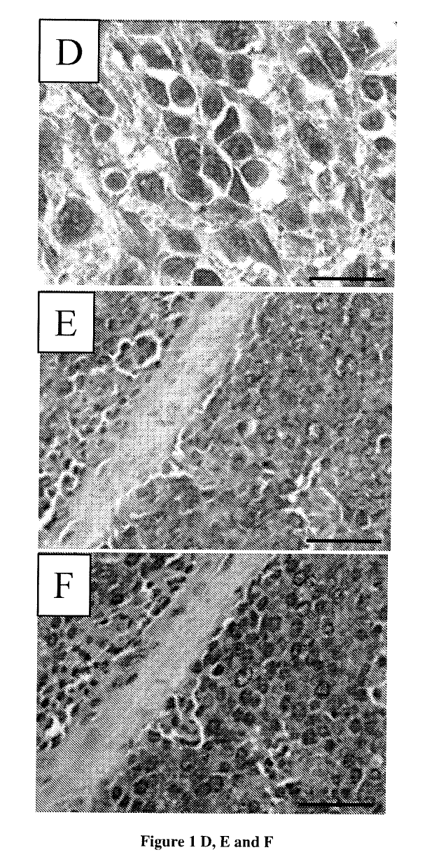

The distribution and localization of OX1R and OxA was investigated on BPH and CaP sections at various stages (Gleason's score 3+3=low grade, 4+3=medium grade and 4+5=high grade). In BPH, OX1R-like immunoreactivity (LI) was confined to scattered cells observed just in a few acini (data not shown). In low and medium grade CaP, some cancerous foci contained cells positive for OX1R (FIG. 1A, B). In high grade CaP, the adenocarcinomatous formations were heavily labelled with the OX1R antibodies (FIG. 1C), and a vast majority of the malignant cells exhibited OX1R-LI (FIG. 1D). In contrast, OX2R was only detected in a few cancer cells only in high grade CaP. Treatment of consecutive sections with either OX1R antibodies, or antibodies directed against the peptide EM66 (which is a marker of neuroendocrine differentiation) revealed that OX1R and EM66 were present in the same carcinomatous formations, whatever the grade of the tumor (FIGS. 1E and F).

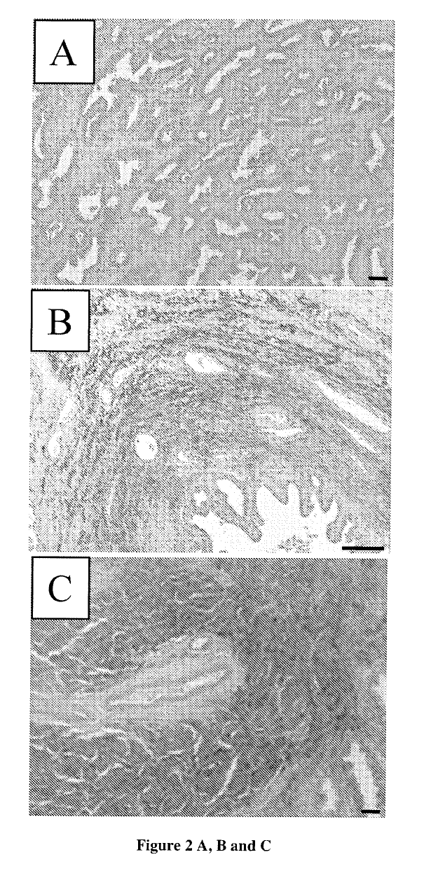

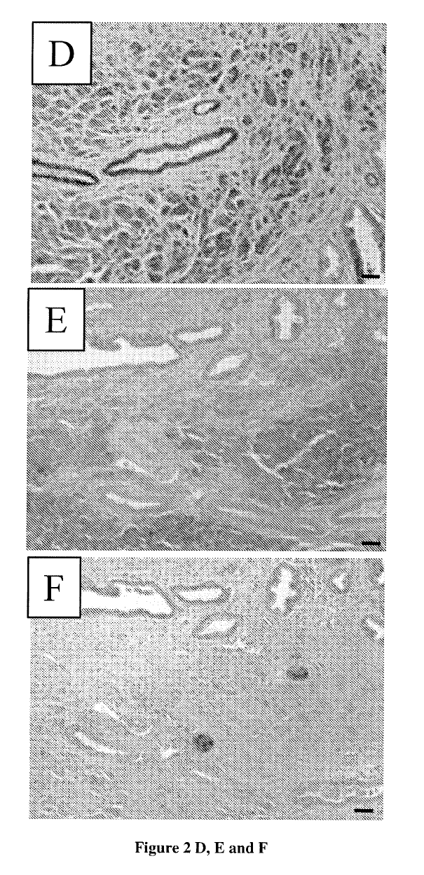

OxA-LI was never observed in cancerous foci whatever the grade of the CaP (FIG. 2A). In contrast, “fiber-like” structures of the stroma were immunostained with the OxA antibodies (FIG. 2B, C, E). Treatment of consecutive sections with the OxA or α-actin (a specific marker of smooth muscle fibers) antibodies revealed that the two antibodies did not label the same structures in the stroma (FIG. 2C, D). Similarly, FIGS. 2E and F show that OxA and PGP 9.5 (a specific marker of nerve fibers) antibodies did not label the same structures.

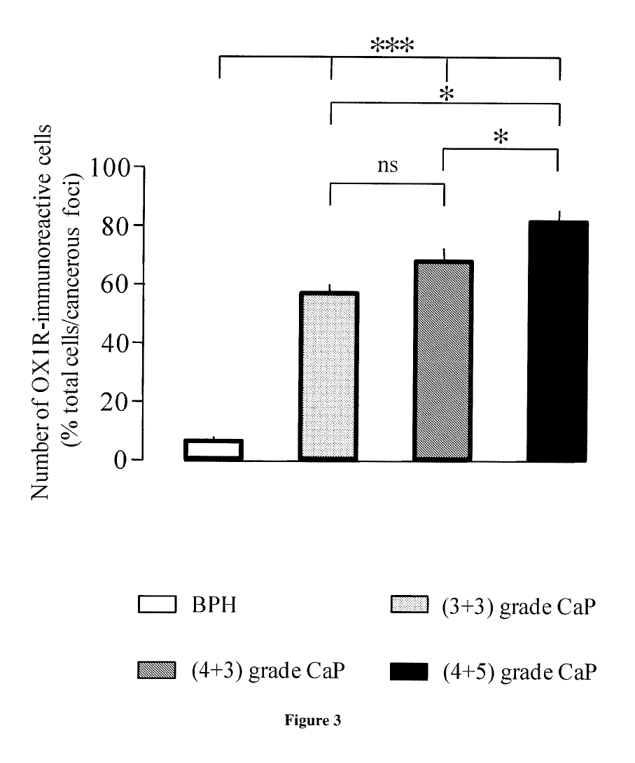

Quantification of immunoreactive cells revealed that the number of OX1R-expressing cells present in the CaP sections was significantly higher (p<0.001) compared to BPH sections (FIG. 3). In addition, the percentage of OX1R-positive cancer cells increased significantly (p<0.05) with the severity of the CaP (FIG. 3).

2.2. Expression of OX1R mRNA in Prostate Cancer Cell Lines

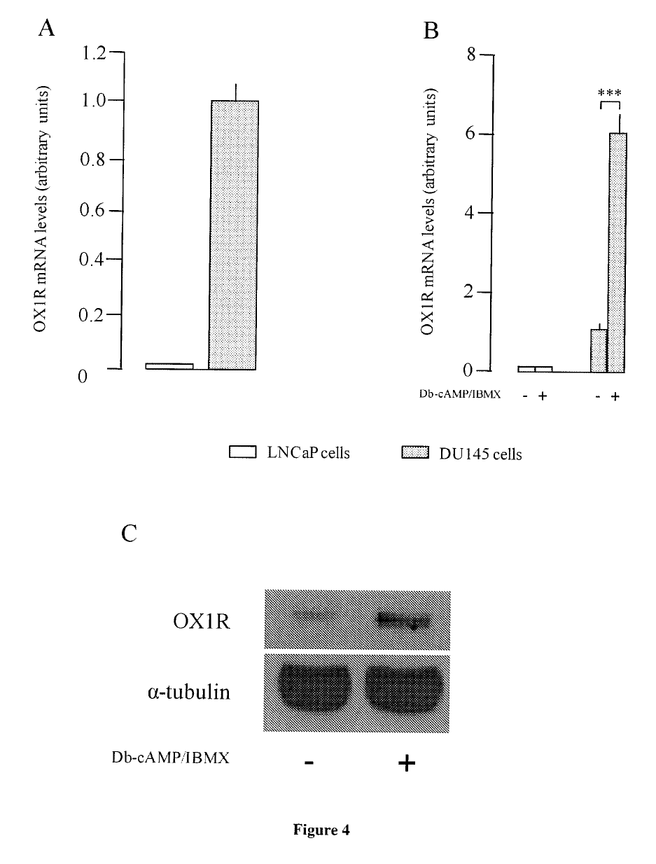

Quantitative RT-PCR performed in two distinct prostate cancer cell lines, i.e. the AD LNCaP and the AI DU145 cells, revealed that the OX1R gene was only expressed in the AI cell line (FIG. 4A). DU145 cells exhibiting a neuroendocrine phenotype showed a highly significant increase of OX1R mRNA expression as compared to the native cells (p<0.001; FIG. 4B), that was confirmed by a western-blot analysis (FIG. 4C). In contrast, induction of a neuroendocrine differentiation in LNCaP cells did not promote the expression of the OX1R gene (FIG. 4B).

2.3. Effects of Orexins on the Apoptosis and Viability of DU145 Cells

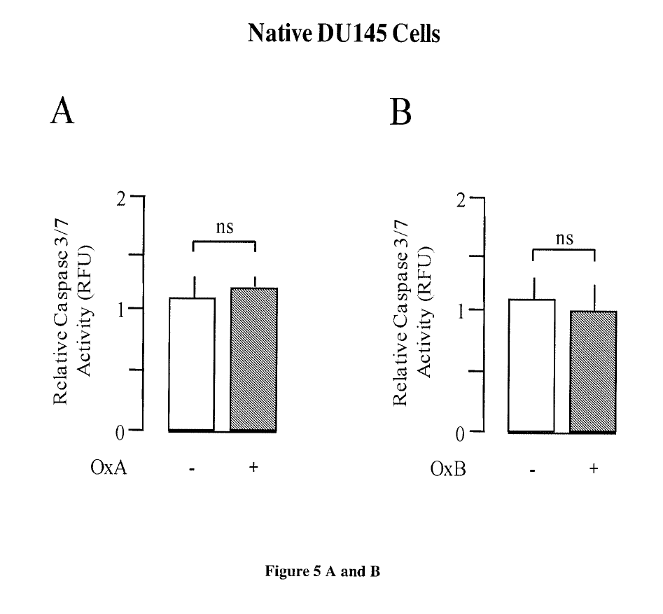

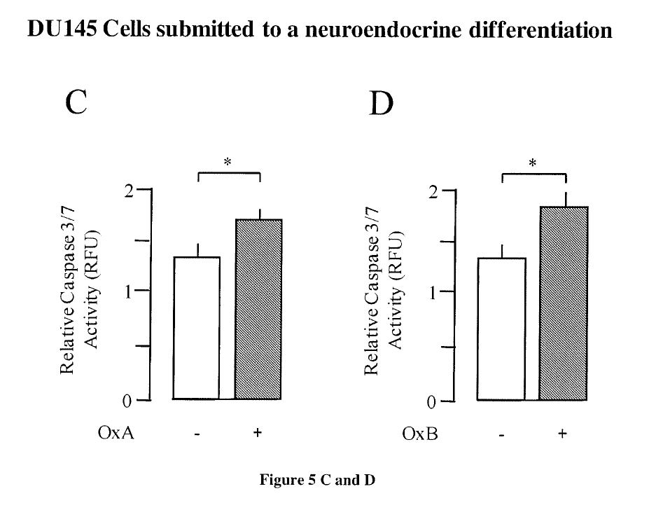

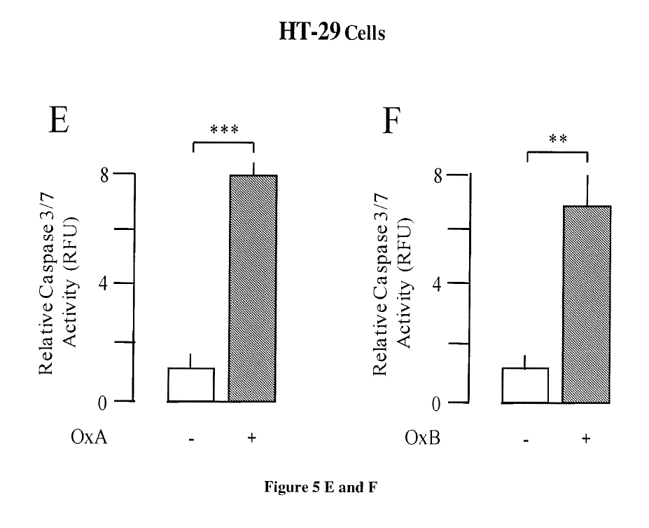

Treatment of native DU145 cells with OxA or OxB (10-6 M each) for 3 days did not alter apoptosis (FIG. 5A, B) but increased cell growth (p<0.05). When the cells were submitted to a neuroendocrine differentiation, incubation with Orexins induced a significant apoptosis (p<0.05; FIG. 5C, D) without altering the number of viable cells. In the same set of experiments, HT-29 cells, used as a positive control, were treated with orexins. As previously described16, OxA and OxB promoted significantly the apoptosis of HT-29 cells (p<0.001 and p<0.01 respectively; FIG. 5E, F) and inhibited cell growth (p<0.05).

2.4. Discussion

This is the first report investigating the presence and function of orexins and their receptors in CaP. The immunohistochemical experiments indicate that the type 1 orexin receptor (OX1R) is present in abundance in carcimatous foci of CaP sections whereas it is virtually absent of non-cancerous prostate tissues. We have performed the same experiments for OX2R and we have found that the orexin type 2 receptor is present in a few cancer cells only in high grade CaP. Such an observation has already been made in colorectal cancer that shows an ectopic expression of the OX1R but not of the OX2R16. We have thus decided to focus our study on OX1R. The quantitative analysis reveals that the number of OX1R-stained cells in the adenocarcinomatous masses increases with the grade of the cancer, and that the orexin receptor is exclusively present in cancerous structures labeled with EM66, a fragment of the granin secretogranin II which has been previously identified as a marker of neuroendocrine differentiation19. Altogether, these findings support the view that the expression of OX1R is closely associated with advanced CaP and the acquisition of a neuroendocrine phenotype by CaP cells. Consistent with this hypothesis, our in vitro data reveal that OX1R is expressed in the AI cell line DU145, but not in the AD cell line LNCaP, and that induction of a neuroendocrine differentiation in the DU145 cells results in an important increase of OX1R expression and production.

The presence of the endogenous ligand of OX1R was also investigated in CaP sections. OxA-LI was detected in “fiber-like” structures of the stroma that do not correspond to smooth muscle fibers or nerve fibers. These OxA-stained structures are probably fibroblasts but this cannot definitely be confirmed as no specific markers of fibroblasts are available. Anyway, in all of the CaP sections treated, we have never observed OxA-labeling in the cancerous foci whatever the grade of the CaP. This observation strongly suggests that OX1Rs expressed by prostate cancer cells might not be activated by a paracrine loop as the stroma structures exhibiting OxA-LI were always located at distance from the cancerous foci. Activation of OX1R in CaP by circulating orexins may be a possibility. However, levels of orexins in human plasma are very low (between 2 and 40 pmol/L) 20 in comparison of the 7 nmol/L Kd of the human OX1R21, making unlikely the activation of OX1R in CaP by circulating orexins. Altogether, our data indicate that OX1R ectopically expressed by prostate cancer cells is probably not activated by endogenous orexins in vivo.

The in vitro experiments reveal that orexins have no effect on the apoptosis of native DU145 cells but stimulate their growth. Consistent with this finding, it has been recently shown that orexin A stimulates INS-1 rat insulinoma cell proliferation via interaction with the OX1R22. In contrast, when the DU145 cells are submitted to a neuroendocrine differentiation, OxA and OxB promote cell death. As trans-differentiated DU145 cells overexpress OX1R, this supports the idea that OX1R level is determinant for the induction of apoptosis as previously suggested for colon cancer cells16. Such an observation has already been reported for the angiotensin II type 2 receptor (AT2R) in hepatocellular carcinoma, in which high levels of AT2R trigger apoptosis while low levels of the receptor do not impact cell death23. The mechanisms underlying OX1R-driven apoptosis in DU145 cells are still unknown. However, it has been shown that transfection of OX1R in CHO cells (devoid of endogenous receptors) is sufficient to confer the ability of orexins to promote apoptosis, and that, in this model as in colon cancer cells, activation of OX1R results in the recruitment and activation of the phosphotyrosine phosphatase SHP-2, and subsequent cytochrome C-mediated mitochondrial apoptosis24. Whether such an intracellular pathway is activated in DU145 cells showing a neuroendocrine phenotype deserves further investigation. The observation that OxA-induced apoptosis increase in DU145 cells with a neuroendocrine phenotype is not associated with a decrease of cell proliferation may be questioning. However, such a phenomenon has already been observed in the same cell line, as it has been reported that inactivation of miR-21 in DU145 cells results in an increase of apoptosis and inhibition of cell motility and invasion, whereas cell proliferation is not affected25.

In conclusion, the present data show that OX1R-driven apoptosis is strongly expressed in AI CaPs showing a neuroendocrine differentiation, opening new perspectives for the treatment of these advanced CaPs which are the most aggressive and for which no treatment is available until now.

### Example 3: In Vivo Results

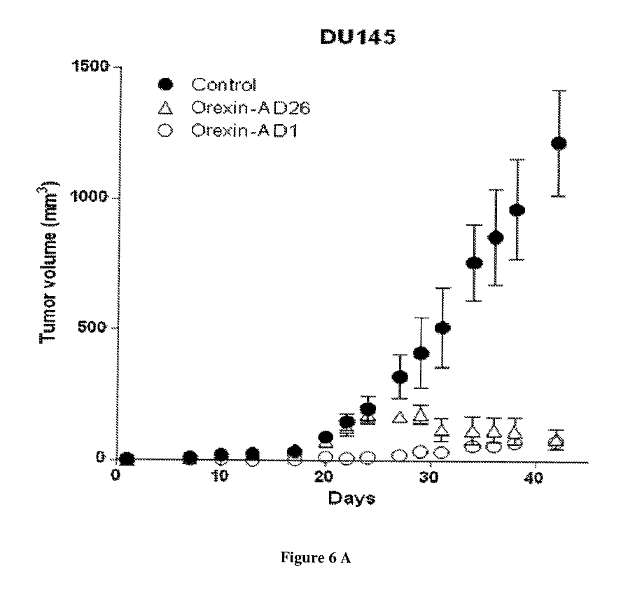

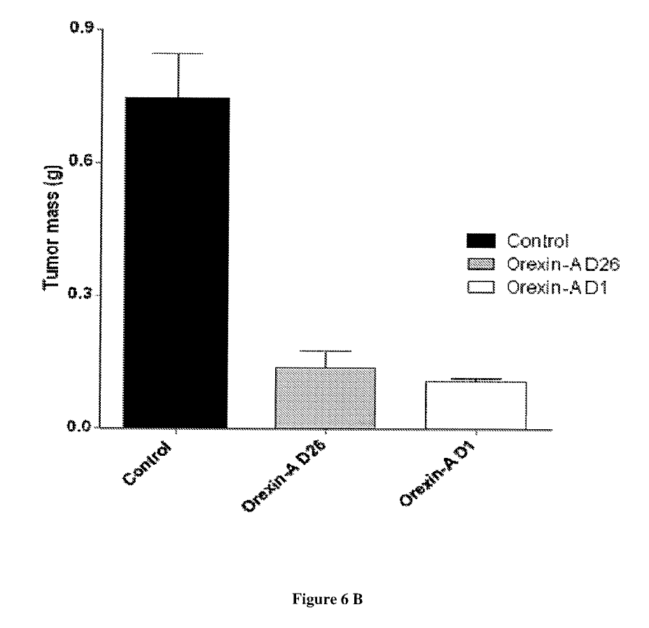

Effect of inoculation of orexin-A on the growth of tumors developed by xenografting human DU-145 cells in nude mice is shown in FIGS. 6 A and B

DU-145 cells were inoculated in the flank of nude mice at day 0. Mice were injected (2 injections/week) intraperitoneally with 100 μl of orexin-A solutions (0.22 μmoles of orexin-A/Kg) starting at day 1 (white circles) or day 26 (white triangles) or with 100 μl of PBS (black circles) for controls (FIG. 6A). After 45 days of treatment, mice were sacrificed and tumor volume and weight were then challenged (FIG. 6B). The development of tumors was followed by calliper measurement. Data are means±SE of 8 tumors in each group. *** p<0.01 versus control.

### REFERENCES

Throughout this application, various references describe the state of the art to which this invention pertains. The disclosures of these references are hereby incorporated by reference into the present disclosure.
1. Nelson E C, Cambio A J, Yang J C, Ok J H, Lara P N Jr., Evans C. Clinical implications of neuroendocrine differentiation in prostate cancer. Prostate Cancer Prostatic Dis 2007; 10, 6-14.
2. Bonkhoff, H. Neuroendocrine cells in benign and malignant prostate tissue: morphogenesis, proliferation, and androgen receptor status. Prostate Suppl 1998; 8, 18-22.
3. Bonkhoff H, Stein U, Remberger K. Endocrine-paracrine cell types in the prostate and prostatic adenocarcinoma are postmitotic cells. Hum Pathol 1995; 26, 167-70.
4. Shariff A H, Ather M H. Neuroendocrine differentiation in prostate cancer. Urology 2006; 68, 2-8.
5. Sumitomo M, Shen R, Walburg M et al. Neutral endopeptidase inhibits prostate cancer cell migration by blocking focal adhesion kinase signaling. J Clin Invest 2000; 106, 1399-407.
6. Ruscica M, Dozio E, Motta M, Magni P. Role of neuropeptide Y and its receptors in the progression of endocrine-related cancer. Peptides 2007; 28, 426-34.
7. Collado B, Sanchez M G, Diaz-Laviada I, Prieto J C, Carmena M J. Vasoactive intestinal peptide (VIP) induces c-fos expression in LNCaP prostate cancer cells through a mechanism that involves Ca2+ signalling. Implications in angiogenesis and neuroendocrine differentiation. Biochim Biophys Acta 2005; 1744, 224-33.
8. Farini D, Puglianiello A, Mammi C, Siracusa G, Moretti C. Dual effect of pituitary adenylate cyclase-activating polypeptide on prostate tumor LNCaP cells: short- and long-term exposure affect proliferation and neuroendocrine differentiation. Endocrinology 2003; 144, 1631-43.
9. Juarranz M G, Bolanos O, Gutierrez-Canas I et al. Neuroendocrine differentiation of the LNCaP prostate cancer cell line maintains the expression and function of VIP and PACAP receptors. Cell Signal 2001; 13, 887-94.
10. Abasolo I, Montuenga L M, Calvo A. Adrenomedullin prevents apoptosis in prostate cancer cells. Regul Pept 2006; 133, 115-22.
11. Rocchi P, Boudouresque F, Zamora A J et al. Expression of adrenomedullin and peptide amidation activity in human prostate cancer and in human prostate cancer cell lines. Cancer Res 2001; 61, 1196-206.
12. Berenguer C, Boudouresque F, Dussert C et al. Adrenomedullin, an autocrine/paracrine factor induced by androgen withdrawal, stimulates ‘neuroendocrine phenotype’ in LNCaP prostate tumor cells. Oncogene 2008; 27, 506-18.
13. Zhong M, Boseman M L, Millena A C, Khan S A. Oxytocin induces the migration of prostate cancer cells: involvement of the Gi-coupled signaling pathway. Mol Cancer Res 2010; 8, 1164-72.
14. Alonzeau J, Alexandre D, Jeandel L et al. The neuropeptide 26RFa is expressed in human prostate cancer and stimulates the neuroendocrine differentiation and the migration of androgeno-independent prostate cancer cells. Europ J Cancer 2013; 49, 511-9.
15. Rouet-Benzineb P, Rouyet-Fessard C, Jarry A et al. Orexins acting at OX1 receptor in colon cancer and neuroblastoma cells or at recombinant OX1 receptor suppress cell growth by inducing apoptosis. J Biol Chem 2004; 279, 45875-86.
16. Voisin T, El Firar A, Fasseu M et al. Aberrant expression of OX1 receptors for orexins in colon cancers and liver metastases: an openable gate to apoptosis. Cancer Res 2011; 71, 3341-51.
17. Voisin T, El Firar A, Avondo V, Laburthe M. Orexin-induced apoptosis: the key role of the seven-transmembrane domain orexin type 2 receptor. Endocrinology 2006; 147, 4977-84.
18. Laburthe M, Voisin T. The orexin receptor OX1R in colon cancer: a promising therapeutic target and a new paradigm in G protein-coupled receptor signaling through ITIMs. Br J Pharmacol 2012; 165, 1678-87.
19. Anouar Y, Demoucelle C, Yon L et al. Identification of a novel secretogranin II-derived peptide (SgII(187-252)) in adult and fetal human adrenal glands using antibodies raised against the human recombinant peptide. J Clin Endocrinol Metab 1998; 83, 2944-51.
20. Heinonen M V, Purhonen A K, Makela K A, Herzig K H. Functions of orexins in peripheral tissues. Acta Physiol 2008; 192, 471-485.
21. El Firar A, Voisin T, Rouyer-Fessard C, Ostuni M A, Couvineau A, Laburthe M. Discovery of a functional immunoreceptor tyrosine-based switch motif in a 7 transmembrane-spanning receptor: role in the orexin receptor OX1R-driven apoptosis. FASEB J 2009; 23, 4069-4080.
22. Chen L, Zhao Y, Zheng D, Ju S, Shen Y, Guo L. Orexin A affects INS-1 rat insulinoma cell proliferation via orexin receptor 1 and the AKT signaling pathway. Int J Endocrinol 2013; 2013, 854623.
23. Du H, Liang Z, Zhang Y et al. Effects of angiotensin II type 2 receptor overexpression on the growth of hepatocellular carcinoma cells in vitro and in vivo. PLoS One 2013; 8, e83754.
24. Laburthe M, Voisin T, El Firar A. Orexins/hypocretins and orexin receptors in apoptosis: a mini-review. Acta Physiol 2010; 198, 393-402.
25. Li T, Li D, Sha J, Huang Y. MicroRNA-21 directly targets MARCKS and promotes apoptosis resistance and invasion in prostate cancer cells. Biochem Biophys Res Commun 2009; 383, 280-285.

1. A method for the treatment of prostate cancer in a subject in need thereof comprising administering the subject with a therapeutically effective amount of a OX1R agonist.

2. The method of claim 1 wherein the OX1R agonist is a small organic molecule.

3. The method of claim 1 wherein the OX1R agonist is an antibody.

4. The method of claim 1 wherein the OX1R agonist is selected from the group consisting of chimeric antibodies, humanized antibodies or full human monoclonal antibodies.

5. The method of claim 1 wherein the OX1R agonist is a polypeptide.

6. The method of claim 1 wherein the OX1R agonist is a functional equivalent of Orexin-A or Orexin-B.

7. The method of claim 1 wherein the OX1R agonist is a polypeptide having at least 80% of identity with SEQ ID NO:2 or 3.

8. The method of claim 1 wherein the OX1R agonist is an immunoadhesin.

9. The method of claim 1 wherein the OX1R is an aptamer.

10. The method of claim 1 wherein the prostate cancer is selected from the group consisting of prostate adenocarcinoma, prostate neuroendocrine tumors, advanced prostate cancer and androgen-independent prostate cancer.

11. The method of claim 1 wherein the prostate cancer is an andregeno-independent prostate cancer.

12. The method of claim 1 wherein the subject is further administered with a chemotherapeutic agent.

13. A method for treating a prostate cancer in a subject in need thereof comprising the steps consisting of i) determining the expression level of OX1R in a tumour tissue sample obtained from the subject, ii) comparing the expression level determined at step i) with a reference value and iii) administering the subject with a therapeutically effective amount of an OX1R agonist when the level determined at step i) is higher than the reference value.

14. A method for screening a drug for the treatment of prostate cancer comprising the steps of i) providing a plurality of test substances ii) determining whether the test substances are OX1R agonists and iii) positively selecting the test substances that are OX1R agonists.


SEQUENCE LISTING (7 sequences)

SEQ ID NO: 1 - protein, 425 aa, Homo sapiens
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPLLFKSTARRARGSILGIWAVSLAIMVPQAAVMECSSVLPELANRTRLFSVCDERWADDLYPKIYHSCFFIVTYLAPLGLMAMAYFQIFRKLWGRQIPGTTSALVRNWKRPSDQLGDLEQGLSGEPQPRGRAFLAEVKQMRARRKTAKMLMVVLLVFALCYLPISVLNVLKRVFGMFRQASDREAVYACFTFSHWLVYANSAANPIIYNFLSGKFREQFKAAFSCCLPGLGPCGSLKAPSPRSSASHKSLSLQSRCSISKISEHVVLTSVTTVLP

SEQ ID NO: 2 - protein, 33 aa, Homo sapiens
  MISC_FEATURE (1)..(1): X is pyruvoglutamate
XPLPDCCRQKTCSCRLYELLHGAGNHAAGILTL

SEQ ID NO: 3 - protein, 28 aa, Homo sapiens
FSGPPGLQGRLQRLLQASGNHAAGILTM

SEQ ID NO: 4 - DNA, 24 bp, Artificial
  Synthetic HPRT1 forward primer
gactttgctttccttggtcaggca

SEQ ID NO: 5 - DNA, 23 bp, Artificial
  Synthetic HPRT1 reverse primer
acaatccgcccaaagggaactga

SEQ ID NO: 6 - DNA, 20 bp, Artificial
  Synthetic OX1R forward primer
gtgggcaacacgctggtctg

SEQ ID NO: 7 - DNA, 20 bp, Artificial
  Synthetic OX1R reverse primer
ggccccagagcttgcggaat